(19) United States
(12) Patent Application Publication
(10) Pub. No.: US 2026/0261911 A1
(43) Pub. Date: Sep. 3, 2026

(54) DYNAMIC OFFLOADING OF UES FROM FR2 UPLINK CARRIERS

(71) Applicant: T-MOBILE INNOVATIONS LLC, Overland Park, KS (US)

(72) Inventors: Timur KOCHIEV, Irvine, CA (US); Mochamad MIRZA, Bothell, WA (US)

(21) Appl. No.: 19/068,237

(22) Filed: Mar. 3, 2025

(51) Int. Cl. H04W 28/082; H04L 5/00; H04W 74/0833

(52) U.S. Cl. CPC H04W 28/082; H04L 5/0044; H04W 74/0833

(57) ABSTRACT

A method for dynamically offloading UEs from frequency range 2 (FR2) carriers is provided. The method includes determining, by a network node, a first UE is not compatible with new radio dual connectivity (NR-DC) and is utilizing a frequency range 2 (FR2) carrier. The method includes determining a second UE is compatible with NR-DC. The method includes predicting, based on real time and historical data, at a first time, a performance of the second UE will be improved using NR-DC if the first UE is offloaded to a frequency range 1 (FR1) carrier. The FR1 carrier is not utilized during NR-DC by the second UE. The method includes instructing, based on the predicting, the first UE to offload the FR2 carrier and instructing the first UE to utilize the FR1 carrier.

### SUMMARY

The present disclosure is directed, in part to systems and methods of dynamically offloading user equipments (UEs) from frequency range 2 (FR2) uplink carriers, substantially as shown and/or described in connection with at least one of the figures, and as set forth more completely in the claims.

Systems and methods for dynamically offloading UEs from FR2 uplink carriers are provided. A non-new radio dual connectivity (NR-DC) compatible device may be instructed to offload a FR2 carrier and instead utilize a frequency range 1 (FR1) uplink carrier that is not being utilized in NR-DC, which frees up available resources of the FR2 carrier such that the NR-DC compatible UEs can use the FR2 carrier for NR-DC. In one example, the determination of whether to offload a UE from a FR2 carrier may be determined based on a prediction of the performance of the NR-DC compatible UE if the non-NR-DC compatible UE is offloaded from the FR2 carrier. Further, additional benefits of NR-DC may be provided by allocating one or more symbols of FR2 signaling channels for data signaling in NR-DC. Such systems and methods provide a dynamic approach that enables NR-DC compatible devices to appreciate the full benefits of NR-DC, enhancing UE performance.

This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

### BRIEF DESCRIPTION OF THE DRAWINGS

FIG. 1 illustrates an exemplary computing device for use with the present disclosure;

FIG. 2 illustrates a diagram of an exemplary network environment in which implementations of the present disclosure may be employed;

FIG. 3 illustrates a flow diagram of an exemplary call flow for an exemplary method for dynamically offloading user equipments (UEs) from frequency range 2 (FR2) uplink carriers in which implementations of the present disclosure may be employed; and

FIG. 4 illustrates a flow diagram of an exemplary method for offloading UEs from FR2 uplink carriers in which implementations of the present disclosure may be employed; and

FIG. 5 illustrates a flow diagram of an exemplary method for offloading UEs from FR2 uplink carriers in which implementations of the present disclosure may be employed.

### DETAILED DESCRIPTION

The subject matter of embodiments of the invention is described with specificity herein to meet statutory requirements. However, the description itself is not intended to limit the scope of this patent. Rather, the inventors have contemplated that the claimed subject matter might be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

Various technical terms, acronyms, and shorthand notations are employed to describe, refer to, and/or aid the understanding of certain concepts pertaining to the present disclosure. Unless otherwise noted, said terms should be understood in the manner they would be used by one with ordinary skill in the telecommunication arts. An illustrative resource that defines these terms can be found in Newton's Telecom Dictionary, (e.g., 32d Edition, 2022). As used herein, the term “base station” refers to a centralized component or system of components that is configured to wirelessly communicate (receive and/or transmit signals) with a plurality of stations (i.e., wireless communication devices, also referred to as user equipment (UE(s))) in a particular geographic area. As used herein, the term “network access technology (NAT)” is synonymous with wireless communication protocol and is an umbrella term used to refer to the particular technological standard/protocol that governs the communication between a UE and a base station; examples of network access technologies include 3G, 4G, 5G, 6G, 802.11x, and the like.

Embodiments of the technology described herein may be embodied as, among other things, a method, system, or computer-program product. Accordingly, the embodiments may take the form of a hardware embodiment, or an embodiment combining software and hardware. An embodiment takes the form of a computer-program product that includes computer-useable instructions embodied on one or more computer-readable media that may cause one or more computer processing components to perform particular operations or functions.

Computer-readable media include both volatile and nonvolatile media, removable and nonremovable media, and contemplate media readable by a database, a switch, and various other network devices. Network switches, routers, and related components are conventional in nature, as are means of communicating with the same. By way of example, and not limitation, computer-readable media comprise computer-storage media and communications media.

Computer-storage media, or machine-readable media, include media implemented in any method or technology for storing information. Examples of stored information include computer-useable instructions, data structures, program modules, and other data representations. Computer-storage media include, but are not limited to RAM, ROM, EEPROM, flash memory or other memory technology, CD-ROM, digital versatile discs (DVD), holographic media or other optical disc storage, magnetic cassettes, magnetic tape, magnetic disk storage, and other magnetic storage devices. These memory components can store data momentarily, temporarily, or permanently.

Communications media typically store computer-useable instructions – including data structures and program modules – in a modulated data signal. The term “modulated data signal” refers to a propagated signal that has one or more of its characteristics set or changed to encode information in the signal. Communications media include any information-delivery media. By way of example but not limitation, communications media include wired media, such as a wired network or direct-wired connection, and wireless media such as acoustic, infrared, radio, microwave, spread-spectrum, and other wireless media technologies. Combinations of the above are included within the scope of computer-readable media.

By way of background, user equipments (UEs), such as cell phones and laptops, may utilize various uplink configurations to communicate with the network. UEs may receive and communicate data utilizing various uplink configurations, such as using new radio dual connectivity (NR-DC). NR-DC, as referred to herein, generally includes a UE connecting to each of a master network node and one or more secondary network nodes, and the UE may utilize a single frequency range 1 (FR1) carrier of the master network node and one or more frequency range 2 (FR2) carriers of the one or more secondary network nodes to achieve increased data speeds and/or increased coverage. Only a limited number of UEs are configured to utilize NR-DC. Systems and methods to optimize the utilization of NR-DC by compatible UEs are valuable.

Conventionally, utilization of the one or more FR2 uplink carriers during NR-DC may provide fewer advantages where default devices not compatible with NR-DC utilize FR2 carriers. For example, a UE may utilize NR-DC to obtain increased performance, however, a large number of non-NR-DC compatible devices are utilizing a majority of FR2 carrier resources. Thus, the improved performance and other advantages of NR-DC are dampened by increased FR2 uplink carrier traffic by non-NR-DC compatible UEs. Approaches that provide, preserve, and/or increase the advantages and benefits of NR-DC are valuable.

In contrast to conventional solutions and to provide a dynamic approach to utilizing uplink configurations, the present disclosure is directed to systems and methods for dynamically offloading UEs from FR2 uplink carriers. Such a non-NR-DC compatible device may instead be instructed to utilize a frequency range 1 (FR1) uplink carrier that is not being utilized in NR-DC, which frees up available resources of the FR2 carrier such that the NR-DC compatible UEs can use the FR2 carrier for NR-DC. The determination of whether to offload a UE from a FR2 carrier may be determined based on a prediction of the performance of the NR-DC compatible UE if the non-NR-DC compatible UE is offloaded from the FR2 carrier, for example. Further, additional benefits of NR-DC may be provided by allocating one or more symbols of FR2 signaling channels for data signaling in NR-DC. Such systems and methods provide a dynamic approach that enables NR-DC compatible devices to appreciate the full benefits of NR-DC, enhancing UE performance.

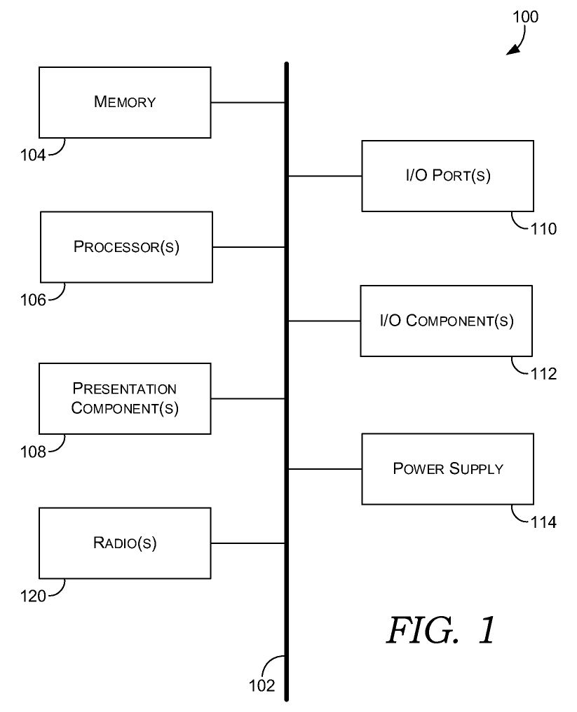

Referring to FIG. 1, an exemplary computer environment is shown and designated generally as computing device 100 that is suitable for use in implementations of the present disclosure. Computing device 100 is but one example of a suitable computing environment and is not intended to suggest any limitation as to the scope of use or functionality of the invention. Neither should computing device 100 be interpreted as having any dependency or requirement relating to any one or combination of components illustrated. In aspects, the computing device 100 is generally defined by its capability to transmit one or more signals to an access point and receive one or more signals from the access point (or some other access point); the computing device 100 may be referred to herein as a user equipment (UE), wireless communication device, or user device. The computing device 100 may take many forms; non-limiting examples of the computing device 100 include a fixed wireless access device, cell phone, tablet, internet of things (IoT) device, smart appliance, automotive or aircraft component, pager, personal electronic device, wearable electronic device, activity tracker, desktop computer, laptop, PC, and the like.

The implementations of the present disclosure may be described in the general context of computer code or machine-useable instructions, including computer-executable instructions such as program components, being executed by a computer or other machine, such as a personal data assistant or other handheld device. Generally, program components, including routines, programs, objects, components, data structures, and the like, refer to code that performs particular tasks or implements particular abstract data types. Implementations of the present disclosure may be practiced in a variety of system configurations, including handheld devices, consumer electronics, general-purpose computers, specialty computing devices, etc. Implementations of the present disclosure may also be practiced in distributed computing environments where tasks are performed by remote-processing devices that are linked through a communications network.

With continued reference to FIG. 1, computing device 100 includes bus 102 that directly or indirectly couples the following devices: memory 104, one or more processors 106, one or more presentation components 108, one or more input/output (I/O) ports 110, one or more I/O components 112, and power supply 114. Bus 102 represents what may be one or more busses (such as an address bus, data bus, or combination thereof). Although the devices of FIG. 1 are shown with lines for the sake of clarity, in reality, delineating various components is not so clear, and metaphorically, the lines would more accurately be grey and fuzzy. For example, one may consider a presentation component such as a display device to be one of the one or more I/O components 112. Also, processors, such as the one or more processors 106, have memory. The present disclosure hereof recognizes that such is the nature of the art, and reiterates that FIG. 1 is merely illustrative of an exemplary computing environment that can be used in connection with one or more implementations of the present disclosure. Distinction is not made between such categories as “workstation,” “server,” “laptop,” “handheld device,” etc., as all are contemplated within the scope of FIG. 1 and refer to “computer” or “computing device.”

Computing device 100 typically includes a variety of computer-readable media. Computer-readable media can be any available media that can be accessed by computing device 100 and includes both volatile and nonvolatile media, removable and non-removable media. By way of example, and not limitation, computer-readable media may comprise computer storage media and communication media. Computer storage media includes both volatile and nonvolatile, removable and non-removable media implemented in any method or technology for storage of information such as computer-readable instructions, data structures, program modules or other data. Computer storage media includes RAM, ROM, EEPROM, flash memory or other memory technology, CD-ROM, digital versatile disks (DVD) or other optical disk storage, magnetic cassettes, magnetic tape, magnetic disk storage or other magnetic storage devices. Computer storage media of the computing device 100 may be in the form of a dedicated solid state memory or flash memory, such as a subscriber information module (SIM). Computer storage media does not comprise a propagated data signal.

Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of any of the above should also be included within the scope of computer-readable media.

Memory 104 includes computer-storage media in the form of volatile and/or nonvolatile memory. Memory 104 may be removable, nonremovable, or a combination thereof. Exemplary memory includes solid-state memory, hard drives, optical-disc drives, etc. Computing device 100 includes one or more processors 106 that read data from various entities such as the bus 102, the memory 104 or the one or more I/O components 112. The one or more presentation components 108 presents data indications to a person or other device. Exemplary one or more presentation components 108 include a display device, speaker, printing component, vibrating component, etc. The one or more I/O ports 110 allow computing device 100 to be logically coupled to other devices including the one or more I/O components 112, some of which may be built in computing device 100. Illustrative I/O components 112 include a microphone, joystick, game pad, satellite dish, scanner, printer, wireless device, etc.

The radio 120 represents one or more radios that facilitate communication with one or more wireless networks using one or more wireless links. While a single radio 120 is shown in FIG. 1, it is expressly contemplated that there may be more than one radio 120 coupled to the bus 102. In aspects, the radio 120 utilizes a transmitted to communicate with a wireless telecommunications network. It is expressly contemplated that a computing device 100 with more than one radio 120 could facilitate communication with the wireless network via both the first transmitter and additional transmitters (e.g. a second transmitter). Illustrative wireless telecommunications technologies include CDMA, GPRS, TDMA, GSM, and the like. The radio 120 may carry wireless communication functions or operations using any number of desirable wireless communication protocols, including 802.11 (Wi-Fi), WiMAX, LTE, 3G, 4G, LTE, 5G, NR, VoLTE, or other VoIP communications. As can be appreciated, in various embodiments, the radio 120 can be configured to support multiple technologies and/or multiple radios can be utilized to support multiple technologies. A wireless telecommunications network might include an array of devices, which are not shown as to obscure more relevant aspects of the invention. Components such as a base station or communications tower (as well as other components) can provide wireless connectivity in some embodiments.

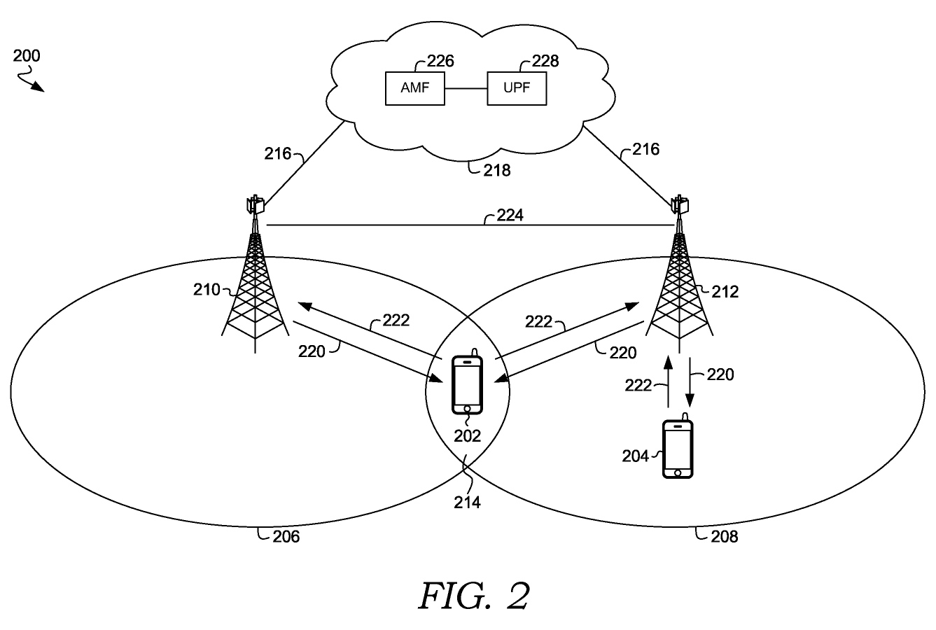

Network environment 200 represents a high level and simplified view of relevant portions of one or more modern wireless telecommunication networks. At a high level, the network environment 200 may generally be said to comprise one or more UEs, such as a first UE 202 and/or a second UE 204, one or more network nodes, such as a first base station 210 and/or a second base station 212, and a network 218, though in some implementations, it may not be necessary for certain features to be present. Similarly, while some components are shown in the singular, it is expressly contemplated that there may be more than one of the components described. The network environment 200 may include a number of routers, switches, and the like. The network environment 200 is generally configured for wirelessly connecting the first UE 202 and/or the second UE 204 to data or services that may be accessible on one or more application servers or other network functions, nodes, or servers not pictured in FIG. 2 so as to not obscure the focus on the present disclosure.

The network environment 200 comprises the first UE 202 and/or the second UE 204. The first UE 202 and the second UE 204 are illustrated as mobile cell phones, however, the first UE 202 and/or the second UE 204 may be internet of things (IoT) devices, fixed wireless access (FWA) devices, laptops, tablets, and the like. While illustrated as specific examples, the first UE 202 and/or the second UE 204 may take any number of forms, including any device discussed with respect to FIG. 1 and may have any one or more components or features of the computing device 100 of FIG. 1. The first UE 202 and/or the second UE 204 may communicate with one or more radio access networks, such as the first base station 210 and/or the second base station 212, to access data and/or services via the network 218. In aspects, the first UE 202 and/or the second UE 204 may be a part of a group of UEs, and may be grouped together based on one or more similarities (e.g., uplink configuration capabilities, location within the network environment 200, associated with subscribers who have purchased a premium subscription). In such aspects, the first UE 202 may be in a different group of UEs than the second UE 204.

The network environment 200 comprises one or more network nodes, such as the first base station 210 and/or the second base station 212, to which the first UE 202 and/or the second UE 204 may potentially connect to (also referred to as ‘camping on,’ ‘attaching,’ in the industry). Though network environment 200 is illustrated with two base stations 210, 212, one skilled in the art will appreciate that more base stations may be present in any particular network environment. While the first base station 210 and/or the second base station 212 is illustrated as the one or more network nodes of the network environment 200, it is expressly contemplated that the one or more network nodes may take other forms (e.g., a satellite). The first base station 210 and/or the second base station 212 of the network environment 200 is generally configured to wirelessly communicate with various UEs, such as the first UE 202 and/or the second UE 204.

In aspects, the first base station 210 may comprise a first coverage area 206 and the second base station may comprise a second coverage area 208, and UEs may obtain access to the network 218 when they are within at least one of the first coverage area 206 and the second coverage area 208. In aspects, the first coverage area 206 and the second coverage area 208 overlap and create an overlapping coverage area 214. In aspects, the first UE 202 is located near a cell edge of the first base station 210, meaning the first UE 202 is at and/or within a threshold distance of an edge of the coverage area 206. In aspects, while the first base station 210 and the second base station 212 are illustrated at different cell sites (e.g., geographic locations) in FIG. 2, the first base station 210 and the second base station 212 may alternatively be co-located at the same cell site. In aspects, the first base station 210 and the second base station 212 may communicate with each other via one or more interfaces (e.g., Xn interface), such as a signaling interface for signaling traffic and a data interface for data traffic.

In the example network environment 200 of FIG. 2, the first UE 202 is shown at the cell edge of each of the first coverage area 206 and the second coverage area 206 and, as a result, may experience reduced signal strength, reduced quality of service, and increased interference. The second UE 204 is shown closer to the second base station 212, relative to the first UE 202, and, as a result, may experience stronger signal strengths and/or resulting high data speeds. The first UE 202 and/or the second UE 204 may move geographically within the coverage areas, which, in some aspects, may impact subsequent predictions or determinations of whether a particular UE, such as the first UE 202 and/or the second UE 204, should be offloaded from a FR2 carrier and/or whether the base stations 210, 212 should allocate symbols of FR2 signaling channels for data signaling. FR2 signaling channels may include a physical uplink control channel (PUCCH) and a random access channel (RACH).

The first base station 210 and/or the second base station 212 may communicate with the first UE 202 and/or the second UE 204 using any wireless telecommunication protocol desired by a network operator, including but not limited to 2G, 3G, 4G, 5G, 6G, 802.11x, LoRa, LoRaWAN, and the like. The first base station 210 and/or the second base station 212 may generally communicate signals to one or more UEs (e.g., the first UE 202 and/or the second UE 204) via a downlink 220 and receive signals from one or more UEs via an uplink 222. In response to receiving certain requests from the first UE 202 and/or the second UE 204, for example, the first base station 210 and/or the second base station 212 may communicate with the network 218 via a backhaul 216. In aspects, the first base station 210 and/or the second base station 212 are configured to receive communications from UEs via FR1 and FR2 uplink carriers, such as the first UE 202 and/or the second UE 204. FR1 carriers generally operate in the sub-7 GHz frequency range (410 MHz to 7125 MHz), making them suitable for wide-area deployments. FR2 carriers generally operate in a higher frequency range (24.25 GHz to 71.0 GHz) in the millimeter wave (mmWave) spectrum, providing high data rates and low latency, but limited coverage and object penetration.

The first UE 202 may utilize one or more compatible uplink configurations, including NR-DC. The first UE 202 is illustrated in FIG. 2 as utilizing NR-DC. In the network environment 200, the first UE 202 communicates, via uplinks 222, to the first base station 212, which is a master base station (e.g., MgNB), and to the second base station 212, which is a secondary base station (e.g., SgNB). The first UE 202 may utilize a FR1 carrier for the uplink 222 to the first base station 210 and the first UE 202 may utilize one or more FR2 carriers for the uplink 222 to the second base station 212. Under this uplink configuration, NR-DC, the first UE 202 leverages wide coverage of an FR1 carrier and the high throughput and data speeds provided by the one or more FR2 carriers. Further, NR-DC may enable the network 218 to mitigate heavy network load and/or network node overloading, as traffic may be distributed across multiple nodes (e.g., the first base station 210, the second base station 212).

The second UE 204 may utilize one or more compatible uplink configurations, which includes utilizing an FR2 carrier for uplink. In aspects, the UE 204 is not compatible with NR-DC. In aspects, such as that shown in the network environment 200, the second UE 204 communicates, via the uplink 222, to a single base station, the second base station 212, using a FR2 uplink carrier. Under these uplink configurations, utilizing an FR2 carrier, the second UE 204 may experience high data rates and low latency, however, the second UE 204 may have less coverage and reliability relative to NR-DC.

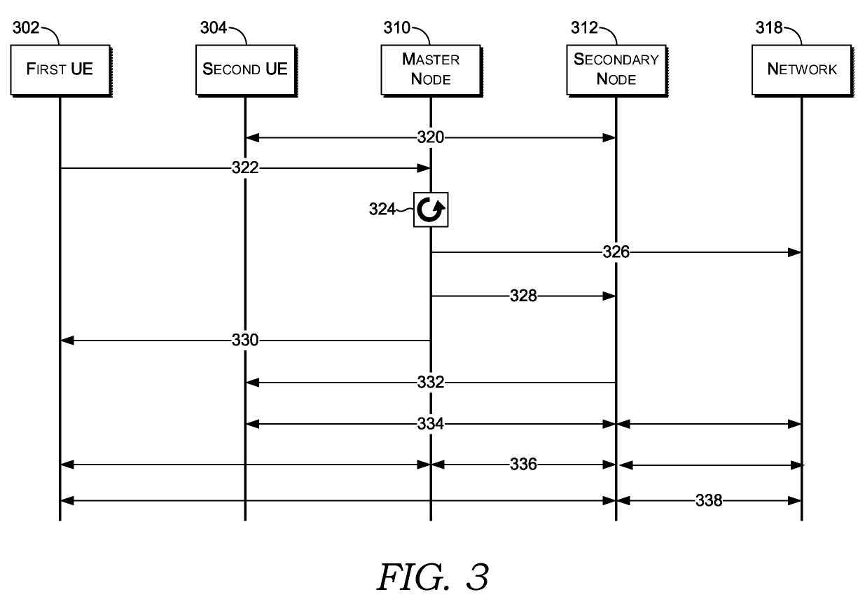

As will be described in more detail with respect to FIG. 3, the first base station 210 and/or the second base station 212 may implement logic to determine one or more determinations or predict one or more predictions based on real time data, such as the time of day in which the network node is determining, and/or historical data, such as a historical indicator of the network load at the time of the day in which the network node is determining.

The network 218 comprises any one or more public or private networks. The network 218 may be configured according to one or more network architectures and/or principles. In some aspects, the network 218 may comprise a cellular telecommunications network (e.g., a 2G, 3G, 4G, 5G, or 6G core network, an IMS network, and the like) and/or a data network (e.g., LAN, WAN, private enterprise network). For example, the network 218 may be a 5G network configured according to new radio (NR) protocols and/or specifications, which may be referred to as a 5G-NR network.

The network 218 may comprise one or more network functions (NFs). As used herein, the term “network function” is used to describe a computer processing module and/or one or more computer executable services being executed on one or more computing processing modules. NFs within the network 218 are defined by their function, as the network 218, in some aspects, may be a service-based architecture. The network 218 may comprise an access and mobility management function (AMF) 226 and a user plane function (UPF) 228. Each of the preceding NFs may take different forms, including consolidated or distributed forms that perform the same general operations. In other architectures or protocols, the NFs may be given other names, however, the NFs herein refer to functions, not specifically identified components. While each of the NFs described above are illustrated in the singular, it is expressly contemplated that the network environment 200 may include one or more of each of the NFs described above.

The AMF 226, for example, is generally responsible for compatibility analysis, facilitating session establishment, and managing the mobility of UEs, such as the first UE 202 and/or the second UE 204, within the network 218. In aspects, the AMF 226 evaluates a UE’s compatibility with the network 218 and/or one or more nodes of the network 218, such as the first base station 210 and/or the second base station 212. The AMF 226 may, in aspects, communicate with a session management function to establish a session with a UE. The UPF 228, for example, is generally responsible for managing user plane traffic. In aspects, the UPF 228 communicates with each of a master network node (e.g., MgNB) and a secondary network node (e.g., SgNB), such as via N3 interfaces, to facilitate user plane traffic routing during an NR-DC uplink configuration.

Turning now to FIG. 3, call flow diagram is illustrated in accordance with one or more aspects of the present disclosure and generally reflects systems and methods for dynamically offloading UEs from FR2 uplink carriers. A call flow 300 may include any one or more aspects described with respect to FIG. 2.

The call flow 300 may generally comprise a first UE 302 (e.g., the first UE 202 of FIG. 2), a second UE 304 (e.g., the second UE 204 of FIG. 2), a master node 310 (e.g., the first base station 210 and/or the second base station 212 of FIG. 2), a secondary node 312 (e.g., the second base station 212 of FIG. 2), and a network 318 (e.g., the network 218 of FIG. 2). While the master node 310 is designated a master node, when the second UE 304 is utilizing the FR2 carrier, the master node 310, may be the only network node the second UE 304 communicates with. The call flow 300 is not meant to exhaustively show every interaction that would be necessary to practice the invention, so as not to obscure the present disclosure. One or more functions and/or components of the network 318 may perform at least some of the functions of the master node 310 and/or the secondary node 312.

At a first step 320, the second UE 204 has an active session with the secondary node 212, and is utilizing a FR2 carrier for uplink to the secondary node 212. The secondary node 212 may determine the second UE 204 is not compatible with NR-DC. In aspects, the secondary node 212 determines the second UE 204 is not compatible with NR-DC based on a group identifier of the second UE 204. In aspects, the group identifier of the second UE 204 is associated with a group of UEs, and each UE within the group of UEs is not compatible with NR-DC. In aspects, the secondary node 212 determines the second UE 204 is not compatible with NR-DC based on compatibility information communicated to the secondary node 212 during establishment of the session of the first step 220.

At a second step 322, the first UE 202 communicates a message or communication to the master node 310. In aspects, the communication from the first UE 302 is an initial communication, such as an attach request. In such aspects, the first UE 302 may provide capability information including compatible uplink configurations, such as NR-DC. In other aspects, the communication to the master node 310 is a communication within an existing session with the network 318, such as to request data and/or services accessible via the network 318. The message may comprise a group identifier of the first UE 302 indicating the first UE 302 is a part of a group of UEs, which may include at least some indicator of the UE’s 302 uplink capabilities. In such aspects, the first UE 302 is in a different group than the second UE 304. For example, the group identifier may comprise an international mobile equipment identity (IMEI) of the first UE 302, which may be associated with the group of UEs and indicate the first UE 302 is a part of the group of UEs. In other aspects, the group identifier is a network slice instance identifier (NSI ID). The first UE 302 may be associated with a group of UEs, where each UE in the group is compatible with NR-DC.

At a third step 324, the master node 310 performs logic to make one or more determinations or predictions. The time at which the third step 324 occurs comprises a first time. The first time may be a time of day, a day of the week, a week of the month, a month of a year, and the like, including any combination thereof. The master node 310 may access real time data associated with the first time and/or historical data to perform the logic of the third step 324. In some aspects, the master node 310 communicates with the secondary node 312 to obtain at least a portion of the real time data and/or the historical data. In aspects, the master node cross-references the real time data with the historical data to predict an uplink utilization suitable for the needs of the UEs 302, 304 (e.g., quality of service, uplink demand) and/or the needs of the master node 310, the secondary node 312, and/or the network 318. The real time data and/or historical data may be stored locally at the master node 310 and/or the secondary node 312 and/or may be accessed by the master node 310 via the network 318. In some aspects, the logic of the third step 324 occurs at regular intervals such that the master node 310 regularly makes the determinations and/or predictions. In aspects, the regular intervals may be every 30 seconds, every minute, every 5 minutes, and the like.

In other aspects, the logic of the third step 324 may be triggered by one or more triggers. The one or more triggers may include the performance of the first UE 302 being suboptimal, the location of the first UE 302 changing, the location of the second UE 304 changing, and/or a change in the load of the network nodes 310, 312 and/or the network 318 generally. In aspects, the logic of the third step 324 is triggered based on a determination by the master node 310 that a performance of the first UE 302 is suboptimal, such as by a performance indicator falling below a pre-determined threshold. In aspects, the performance indicator may be a reference signal received power (RSRP) value, a signal to noise interference ratio (SINR) value, a received signal strength indicator (RSSI) value, a reference signal received quality (RSRQ) value, channel quality indicator (CQI) value, and the like. In aspects, the network load within the network 318 may change such that an indicator of the network load (e.g., throughput, latency, packet loss) indicates the network is congested (e.g., indicator exceeds a threshold) and/or the network nodes 310, 312 are overloaded (e.g., indicator exceeds a threshold) and triggers the logic of the third step 324. In aspects, the location of the first UE 302 and/or the second UE 304 may change such that the first UE 302 and/or the second UE 304 is within a threshold distance of the cell edge and/or is not within the threshold distance of the cell edge, which triggers the logic of the third step 324. In aspects, the trigger may comprise the occurrence of a historical time in which the network load is high or low, a historical time in which the first UE 302 and/or the second UE 304 has high or low uplink demand, and the like.

The real time data utilized by the master node 310 at the third step 324 may include performance of the first UE 302, performance of the second UE 304, network load information, location information associated with the first UE 302 and/or the second UE 304, and/or a time in which the third step 324 occurs (i.e., the first time). The real time data may include the performance of the first UE 302 at the first time (e.g., RSRP, SINR, RSSI, RSRQ) and the performance of the second UE 304 at the first time. The real time data may include an indication of the network load of the network 318 generally (e.g., throughput, latency, packet loss) at the first time. For example, the indicator of the network load may indicate the network is congested and/or that a network node is overloaded. The real time data may include location information of the first UE 302 and/or the second UE 304 at the first time. The location information may comprise timing advance (TA), round trip time (RTT), angle of arrival (AoA), time difference of arrival (TDOA), and the like. In aspects, the location information comprises a determination that the first UE 302 and/or the second UE 304 is within the threshold distance of a cell edge of the master node 310. In aspects, when the first UE 302 is within the threshold distance of the cell edge, the first UE 302 may, in some aspects based on the real time data and/or the historical data, have improved performance when utilizing an NR-DC uplink configuration. The real time data may include a utilization of one or more FR2 signaling channels at the first time (e.g., resource block utilization, packet count over time, throughput).

The historical data utilized by the master node 310 at the third step 324 may include an uplink utilization pattern of the first UE 302 and/or the second UE 304 during at a particular time of day, day of the week, month of the year, and the like, including any combination thereof. For example, the first UE 302 may have low uplink demand from the hours of 5PM to 9PM within a singular day, and may have high uplink demand from the hours of 9AM to 12PM within the singular day, and the second UE 304 may have low uplink demand from the hours of 5PM to 9PM, and high uplink demand from the hours of 10AM to 4PM. This pattern may vary depending on the day of the week, for example. The historical data may include historical location data of the first UE 302 and/or the second UE 304, such as a most frequent location (e.g., the location in which the first UE 302 and/or the second UE 304 is present the longest during a given time period). In aspects, the historical data may include the network’s 308 utilization pattern during a time period. For example, the network 318 may be under-utilized from 12AM to 5AM during a Saturday 24 hour period, which may impact the determination at the third step 324. The historical data may include performance history data of the first UE 302, the second UE 304, and/or the network 318. In aspects, the master node 310 may log performance indicators of the first UE 302 and/or the second UE 304, the first UE’s 302 location relative to the master node 310 (e.g., within or outside of a threshold distance from the cell edge), the second UE’s 304 location relative to the secondary node 312, the first UE’s 302 and/or the second UE’s 304 particular uplink configuration (e.g., NR-DC, utilizing a single FR2 carrier), the first UE’s 302 and/or the second UEs 304 uplink demand, the utilization of the FR2 signaling channels, and/or one or more indicators of network load (e.g., of the master node 310, of the network 318) collected at one or more times prior to the first time by the master node 310.

In aspects, the first UE 302 and/or the second UE 304 may each be associated with a group of UEs. In aspects, the first UE 302 is associated with a first group and the second UE 304 is associated with a second group. In some aspects, the group of UEs are subscribed to the same network slice such that the master node 310 only performs the logic of the third step 324 for UEs subscribed to a particular network slice. In other aspects, the group of UEs is an IMEI group, an IMEI software version (IMEI SV) group, an internet protocol (IP) address group, media access control (MAC) address group, subscriber identifier group, and the like. The group of UEs may be grouped based on any one or more similarities shared among each UE within the group. In aspects, the group of UEs may be grouped based on their compatibility with an NR-DC or their incompatibility with an NR-DC uplink configuration. Conventionally, very few UEs are compatible with utilizing NR-DC for uplink. In one example, the first UE 302 may be compatible with NR-DC, and may be grouped based on this capability. In another example, the second UE 304 may not be compatible with NR-DC and may be grouped based on this lack of compatibility. The group of UEs may be grouped based on their most frequent locations (e.g., a location in which the first UE 302 is present the longest during a given time period, such as a month) being within the threshold distance of the cell edge of the master node 310 and/or the secondary node 312. In aspects, the group of UEs corresponding to the group of UEs compatible with NR-DC may be prioritized for the methods described herein such that only UEs within this group may be eligible to receive the methods described herein.

In some aspects, at the third step 324, the master node 310 utilizes artificial intelligence and/or machine learning principles to make one or more determinations or predictions. The master node 310 may consider the performance of the first UE 302 and/or the second UE 304 and/or the uplink demand of the first UE 302 with the uplink demand of the second UE 304 and/or the network load of the nodes 310, 312 and/or the network 318, based on the real time data at the first time and the historical data. In aspects, the master node 310 uses the performance history logs of the first UE 302 and/or the second UE 304 to, based on the real time data of the at the first time to make the one or more determinations or predictions.

The one or more determinations or predictions may include a prediction that a UE’s performance will be improved when one or more UEs alter their uplink configuration. In one example, the first UE 302 is predicted to have improved performance (e.g., relative to the performance of the first UE 302 at the first time) if the second UE 304 is offloaded from the FR2 carrier to a FR1 carrier. In aspects, the FR1 carrier is not utilized during NR-DC. In aspects, the first UE 302 may utilize the FR2 carrier in which the second UE 304 was offloaded from for NR-DC. In another example, the second UE 304 may be located within a threshold distance of the cell edge, and may be experiencing poor coverage due to utilizing a single FR2 carrier. In this example, the master node 310 may predict the performance of each of the first UE 302 and the second UE 304 may be improved if the second UE 304 is offloaded from the FR2 carrier and utilizes a FR1 carrier not utilized during NR-DC, as a FR1 carrier may provide greater coverage near the cell edge compared to the FR2 carrier. Further, the master node 310 may determine the performance of the first UE 302 may improve by offloading the second UE 302 from the FR2 carrier, as the first UE 302 can utilize the FR2 carrier for NR-DC.

The one or more determinations or predictions may include a prediction of one or more utilizations of one or more FR2 signaling channels, such as whether the utilization is high or low (relative to a designated baseline). In one example, the master node 310 may predict that the utilization of one or more FR2 signaling channels will be low at one or more times subsequent to the first time. For example, the historical data may indicate the FR2 signaling channels have low utilization during the hours of 9PM to 1AM, and the first time may be at 10PM. In some aspects, the utilization of the one or more FR2 signaling channels is predicted to be low, based on the real time and the historical data, and based on the fact that offloading the second UE 302 to the FR1 carrier frees up the FR2 carrier for NR-DC. Based on the prediction, the master node 310 may determine to allocate one or more symbols of the one or more FR2 signaling channels for data signaling, which may be utilized by the first UE 302 in an NR-DC uplink configuration. In such aspects, the first UE 302 may utilize the one or more symbols using a physical uplink shared channel (PUSCH).

In another example, the historical data may indicate the first UE 302 has low uplink demand from the hours of 9PM-5AM, and the second UE 304 has high uplink demand from the hours of 10PM to 12AM. The real time data may indicate the first time is 10PM, and that the second UE 304 is near the secondary base station 312. In aspects, the master node 310 may predict that the performance of the first UE 302 may improve using NR-DC for uplink. However, given the predicted reduced uplink demand of the first UE 302 and the predicted increased uplink demand of the second UE 304, the master node 310 may determine to not instruct the second UE 304 to offload the FR2 carrier. In this example, the call flow 300 may cease, and the first UE 302 and the second UE 304 may continue using their existing uplink configurations utilized at the first step 220 and the second step 222.

At a fourth step 326, the master node 310 communicates the determination or prediction determined or predicted by the master node 310 at the third step 324 to the network 318. In aspects, the master node 310 communicates the determination or prediction to an AMF (e.g., the AMF 226 of FIG. 2) of the network 318, which may modify a session between the first UE 302 and the network 318 and/or the second UE 304 and the network 318 via the master node 310 and/or the secondary node 312.

At a fifth step 328, the master node 310 may communicate the determination or prediction to the secondary node 312, such that the secondary node 312 may instruct for a reconfiguration of the second UE 304 based on the determination or prediction by the master node 310 in the third step 324. In aspects, the master node 310 may inform the secondary node 312 that it will be allocating one or more symbols of the signaling channels of the master node 310 for data signaling by the first UE 302 during NR-DC. In aspects, the master node 310 may instruct the secondary node 312 to allocate one or more symbols of the signaling channels for data signaling by the first UE 302 during NR-DC.

In aspects, at a sixth step 330, the master node 310 may instruct the first UE 302 to utilize a modified uplink configuration, which may have been determined or predicted by the master node 310 at the third step 324, and the first UE 302 receives the instructions to utilize the modified uplink configuration determined at the third step 324. In some aspects, the uplink configuration determined at the third step 324 is the same as the uplink configuration the first UE 302 utilized to send the communication of the first step 320 (i.e., the second UE 304 is not offloaded to the FR2 carrier). In other aspects, the uplink configuration determined at the third step 324 is different than the uplink configuration the first UE 302 utilized to send the communication of the first step 320. In some aspects, the master node 310 may instruct, based on the determination or prediction, the UE 302 to utilize the FR2 carrier that the second UE 304 is being offloaded from. In aspects, the master node 310 utilizes radio resource control (RRC) messages to instruct the first UE 302.

In aspects, at a seventh step 332, the secondary node 312 may instruct the second UE 304 to utilize a modified uplink configuration, which may have been determined or predicted by the master node 310 at the third step 324. In aspects, the secondary node 312 instructs the second UE 304 to offload the FR2 carrier such that the first UE 302 may effectively utilize the FR2 carrier during NR-DC. In aspects, at the seventh step 332, the secondary node 312 allocates the one or more symbols of the one or more FR2 signaling channels for data signaling by the first UE 302 using NR-DC.

In aspects, at an eighth step 334, the second UE 304 re-configures its uplink configuration according to the modified uplink configuration to establish a connection with the secondary node 312. In aspects, the second UE 304 utilizes an FR1 carrier in the session of the eighth step 334, as it has been offloaded from the FR2 carrier utilized in the session of the first step 320. In such aspects, the single FR1 carrier is not to be utilized in NR-DC. The master node 310 may communicate with the network 318 to enable access to data and/or services to the second UE 304.

In aspects, at a ninth step 336, the first UE 302 communicates with the master node 310 utilizing the modified uplink configuration determined in the third step 324, and the master node 310 communicates with the network 318 to enable access to data and/or services to the first UE 302. In aspects, the first UE 302 communicates with the master node 310 using a single FR1 carrier. At a tenth step 338, the first UE 302 communicates with the secondary node 312 utilizing at least the FR2 carrier upon which the second UE 304 is offloaded from, and the secondary node 312 communicates with the network 318 to enable access to data and/or services to the first UE 302

In aspects, at a second time occurring subsequent to the first time, the master node 310 may re-determine or re-predict, based on real time data associated with the second time and/or historical data, the one or more determinations or predictions described with respect to the third step 324. In aspects, based on the re-determination or re-prediction, the master node 310 may determine to instruct the first UE 302 to cease utilizing the modified uplink configuration and/or instruct the second UE 304 (e.g., via the secondary node 312) to cease utilizing the single FR1 carrier. In such aspects, the master node 310 may instruct the first UE 302 and/or the second UE 304 to utilize different uplink configurations based on the re-determination (e.g., the uplink configurations utilized at the first step 320 and the second step 322). Further, at the second time, the master node 310 may determine, based on the re-determination or re-prediction, to allocate the one or more symbols of the FR2 signaling channels back to the FR2 signaling channels. In such aspects, the master node 310 may instruct the first UE 302 to cease data signaling using the FR2 signaling channels such that the one or more FR2 signaling channels can communicate signaling traffic. In aspects, the re-determination or re-prediction is one of a regular interval of re-determinations and/or is triggered by one or more triggers described with respect to the third step 324.

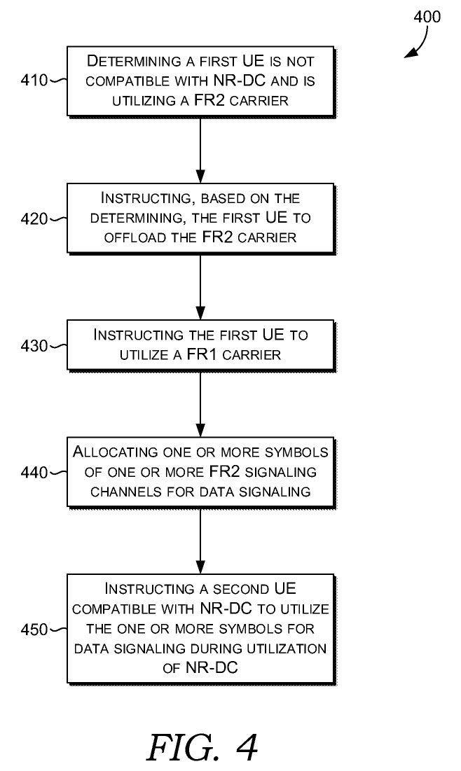

Now referring to FIG. 4, a flow chart is provided that illustrates one or more aspects of the present disclosure relating to a method 400 for dynamically offloading UEs from FR2 uplink carriers. The method 400 may include any one or more aspects described with respect to FIGS. 2-3.

At a first step 410, the method 400 includes determining, by a network node (e.g., the first base station 210 and/or the second base station 212 of FIG. 2, the master node 310 and/or the secondary node 312 of FIG. 3), a first user equipment (UE) (e.g., the second UE 204 of FIG. 2, the second UE 304 of FIG. 3) is not compatible with of new radio dual connectivity (NR-DC) and is utilizing a FR2 carrier for uplink. In aspects, the first UE is a part of the group of UEs, and each UE of the group of UEs is not compatible with NR-DC. At a second step 420, the method 400 includes instructing, based on the determining, the first UE to offload the FR2 carrier, as described with respect to FIG. 3. In aspects, the instructing is based on a prediction occurring at a first time, predicted based on real time and historical data, that a performance of the first UE will not be impacted by offloading the FR2 carrier (e.g., a performance indicator of the first UE at the first time is within a threshold of a predicted performance indicator of the first UE).

At a third step 430, the method 4000 includes instructing the first UE to utilize a FR1 carrier. In aspects, the FR1 carrier is not utilized during NR-DC. At a fourth step 440, the method 400 includes allocating one or more symbols of one or more FR2 signaling channels for data signaling. In aspects, the allocating may be based on a prediction, predicted based on the real time and historical data, that a utilization of one or more FR2 signaling channels is predicted to be low at one or more times subsequent to a time of the third step 430 (e.g., the first time).

At a fifth step 450, the method 400 includes instructing a second UE (e.g., the first UE 202 of FIG. 2, the first UE 302 of FIG. 3) compatible with NR-DC to utilize the one or more symbols for data signaling during utilization of NR-DC. In aspects, the network node determines the second UE is compatible with NR-DC based on the second UE being a part of a group of UEs, where each UE in the group is compatible with NR-DC. In aspects, the second UE utilizes the one or more symbols using a physical uplink shared channel (PUSCH).

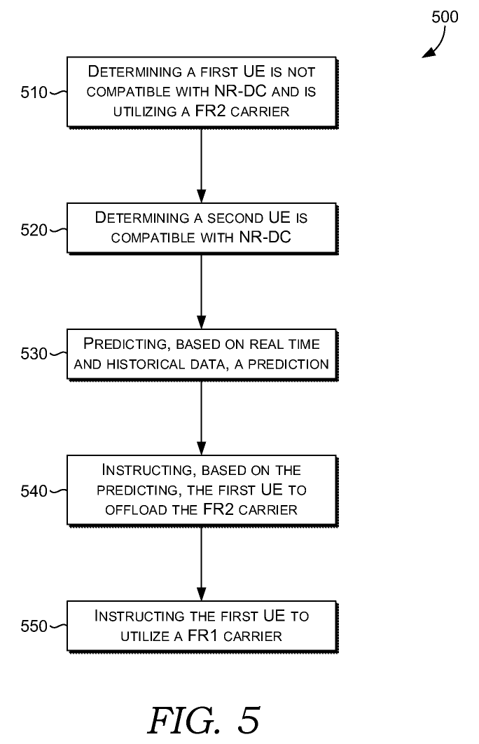

Now referring to FIG. 5, a flow chart is provided that illustrates one or more aspects of the present disclosure relating to a method 500 for dynamically offloading UEs from FR2 uplink carriers. The method 500 may include any one or more aspects described with respect to FIGS. 2-4.

At a first step 510, the method 500 includes determining, by a network node, a first UE (e.g., the second UE 202 of FIG. 2, the second UE 302 of FIG. 3) is not compatible with NR-DC and is utilizing an FR2 carrier, as described herein. At a second step 520, the method 500 includes determining a second UE (e.g., the first UE 202 of FIG. 2, the first UE 304 of FIG. 3) is compatible with NR-DC.

At a third step 530, the method 500 includes predicting, at a first time, based on real time and historical data, a prediction. In aspects, the prediction is that a performance of the second UE will be improved using NR-DC if the first UE is offloaded to a FR1 carrier. In such aspects, the FR1 carrier is not utilized during NR-Dc by the second UE. In aspects, the prediction is that a utilization of one or more FR2 signaling channels is predicted to be low at one or more times subsequent to the first time. In aspects, the prediction is that a performance of the first UE will be improved or at least not impacted by offloading the FR2 carrier.

At a fourth step 540, the method 500 includes instructing, based on the predicting, the first UE to offload the FR2 carrier. At a fifth step 550, the method includes instructing the first UE to utilize a FR1 carrier. In aspects, the FR1 carrier is not utilized during NR-DC, whether by the second UE or other NR-DC utilizing UEs. The method 500 may further include instructing the second UE to utilize the FR2 carrier upon which the first UE has been offloaded from. The method 500 may further include instructing the first UE to utilize the one or more symbols of the one or more FR2 signaling channels for data signaling during NR-DC.

Many different arrangements of the various components depicted, as well as components not shown, are possible without departing from the scope of the claims below. Embodiments in this disclosure are described with the intent to be illustrative rather than restrictive. Alternative embodiments will become apparent to readers of this disclosure after and because of reading it. Alternative means of implementing the aforementioned can be completed without departing from the scope of the claims below. Certain features and subcombinations are of utility and may be employed without reference to other features and subcombinations and are contemplated within the scope of the claims

In the preceding detailed description, reference is made to the accompanying drawings which form a part hereof wherein like numerals designate like parts throughout, and in which is shown, by way of illustration, embodiments that may be practiced. It is to be understood that other embodiments may be utilized and structural or logical changes may be made without departing from the scope of the present disclosure. Therefore, the preceding detailed description is not to be taken in the limiting sense, and the scope of embodiments is defined by the appended claims and their equivalents.

We claim:

1. A system for dynamically offloading UEs from frequency range 2 (FR2) uplink carriers, the system comprising:
one or more computer processing components configured to perform operations comprising:
determining, by a network node, a first UE is not compatible with new radio dual connectivity (NR-DC) and is utilizing a frequency range 2 (FR2) carrier;
determining a second UE is compatible with NR-DC;
predicting, based on real time and historical data, at a first time, a performance of the second UE will be improved using NR-DC if the first UE is offloaded to a frequency range 1 (FR1) carrier, wherein the FR1 carrier is not utilized during NR-DC by the second UE;
instructing, based on the predicting, the first UE to offload the FR2 carrier; and
instructing the first UE to utilize the FR1 carrier.

2. The system of claim 1, wherein the real time data comprises a time of day of the first time, and wherein the historical data comprises a historical uplink demand of the second UE at the time of day of the first time.

3. The system of claim 1, wherein the real time data comprises a performance of the second UE at the first time, and wherein the historical data comprises a historical performance of the second UE using NR-DC.

4. The system of claim 1, wherein the real time data comprises a location of the second UE at the first time, and wherein the historical data comprises a historical performance of the second UE using NR-DC at the location.

5. The system of claim 1, wherein determining the first UE is not compatible with NR-DC is determined based off an international mobile equipment identify (IMEI) of the first UE being associated with a group of UEs that are not compatible with NR-DC.

6. The system of claim 1, further comprising predicting, based on the real time data and the historical data, a utilization of FR2 signaling channels is predicted to be low subsequent to the first time.

7. A method for dynamically offloading UEs from frequency range 2 (FR2) uplink carriers comprising:
determining, by a network node, at a first time, a first UE is not compatible with new radio dual connectivity (NR-DC) for uplink and is utilizing a frequency range 2 (FR2) carrier;
instructing, based on the determining, the first UE to offload the FR2 carrier;
instructing the UE to utilize a frequency range 1 (FR1) carrier, wherein the FR1 carrier is not used with NR-DC;
allocating one or more symbols of one or more FR2 signaling channels for data signaling; and
instructing a second UE compatible with NR-DC to utilize the one or more symbols for data signaling during utilization of NR-DC.

8. The method of claim 7, wherein the one or more FR2 signaling channels comprise a physical uplink control channel (PUCCH) and a random access channel (RACH).

9. The method of claim 7, wherein determining the first UE is not compatible with NR-DC is determined based off an international mobile equipment identify (IMEI) of the first UE being associated with a group of UEs that are not compatible with NR-DC.

10. The method of claim 7, wherein the allocating is based on a prediction that a utilization of the one or more FR2 signaling channels will be low at one or more times subsequent to the first time.

11. The method of claim 7, further comprising determining the second UE is compatible with NR-DC based off an international mobile equipment identify (IMEI) of the second UE being associated with a group of UEs that are compatible with NR-DC.

12. The method of claim 7, wherein the second UE utilizes the one or more symbols using a physical uplink shared channel (PUSCH).

13. A method for dynamically offloading UEs from frequency range (FR2) uplink carriers comprising:
determining, by a network node, a first UE is not compatible with new radio dual connectivity (NR-DC) and is utilizing a frequency range 2 (FR2) carrier;
determining a second UE is compatible with NR-DC;
predicting, based on real time and historical data, at a first time, a utilization of one or more FR2 signaling channels is predicted to be low at one or more times subsequent to the first time;
instructing, based on the predicting, the first UE to offload the FR2 carrier;
instructing the first UE to utilize a FR1 carrier, wherein the FR1 carrier is not utilized during NR-DC; and
instructing the second UE to utilize the one or more symbols of the one or more FR2 signaling channels for data signaling during NR-DC.

14. The method of claim 13, wherein the one or more FR2 signaling channels comprise a physical uplink control channel (PUCCH) and a random access channel (RACH).

15. The method of claim 13, wherein determining the first UE is not compatible with NR-DC is determined based off an international mobile equipment identify (IMEI) of the first UE being associated with a group of UEs that are not compatible with NR-DC.

16. The method of claim 13, further comprising allocating the one or more symbols of the one or more FR2 signaling channels for data signaling.

17. The method of claim 13, further comprising determining the second UE is compatible with NR-DC based off an international mobile equipment identify (IMEI) of the second UE being associated with a group of UEs that are compatible with NR-DC.

18. The method of claim 7, wherein the second UE utilizes the one or more symbols using a physical uplink shared channel (PUSCH).

19. The method of claim 7, wherein the real time data comprises a time of day of the first time, and wherein the historical data comprises a historical FR2 signaling channel utilization at the time of day of the first time.

20. The method of claim 7, further comprising predicting that a performance of the second UE will be improved by utilizing NR-DC.
